(19) United States
(12) Patent Application Publication
(10) Pub. No.: US 2014/0364359 A1
(43) Pub. Date: Dec. 11, 2014

(54) COMPOSITIONS AND METHODS FOR TREATING CANCER AND INFLAMMATION-RELATED DIESEASES AND CONDITIONS

(71) Applicant: Raymond A FIRESTONE, (US)

(72) Inventor: Raymond A. Firestone, New York, NY (US)

(21) Appl. No.: 14/372,607

(22) PCT Filed: Jan. 17, 2013
(86) PCT No.: PCT/US2013/021816

§ 371 Date: Jul. 16, 2014

Related U.S. Application Data
(60) Provisional application No. 61/588,154, filed on Jan. 18, 2012.

(51) Int. Cl. A61K 47/48

(52) U.S. Cl. CPC A61K 47/48038; A61K 47/48246
USPC 514/1.7; 536/6.4; 514/19.3; 514/19.4; 514/19.5; 514/19.6; 514/21.91; 514/16.6; 514/1.9; 514/16.8; 514/6.9

(57) ABSTRACT

The invention provides di-peptide conjugated antitumor agents, pharmaceutical compositions and methods for preparation and use thereof for treating various cancer and inflammation-related diseases and conditions. The present invention addresses the shortcomings of the existing anti-tumor and anti-inflammatory drugs, particularly in that the anti-tumor agents of the invention that selectively kill cancer cells with minimal damage to normal cells. Similarly, the anti-inflammatory agents of the invention provide effective treatment of various inflammatory diseases and conditions without the many deleterious side effects commonly associated with steroids

### PRIORITY CLAIMS AND RELATED PATENT APPLICATIONS

This application is the national phase of PCT/US13/21816, filed Jan. 17, 2013, which claims the benefit of priority from U.S. Provisional Application Ser. No. 61/588,154, filed on Jan. 18, 2012, the entire content of which is incorporated herein by reference in its entirety.

### TECHNICAL FIELDS OF THE INVENTION

The invention generally relates to novel compounds, compositions and methods of therapeutic treatment of various cancer and inflammatory diseases and conditions. More particularly, the invention relates to di-peptide conjugated antitumor agents, methods for their preparation, and pharmaceutical compositions and uses thereof, especially in treating cancer and inflammation-related diseases and conditions.

### BACKGROUND OF THE INVENTION

Despite decades of intensive scientific and clinical research, cancer remains a major health threat to the public. While significant advancements have been made in cancer prevention and treatment, cancer remains a challenging disease to both the patient and the healthcare provider. It is estimated that in the U.S. alone, there are over 1.5 million new cases of cancer and more than half million of cancer-related deaths in 2011. Globally, cancer is the third leading cause of death.

Cancer is characterized by rapidly-proliferating cell growth in the body. Cancer is often able to invade other tissues from its original location and, in a process called metastasis, spread to other parts of the body through blood and lymphatics. There are many types of cancer, which may be classified in pathology and clinical diagnosis into carcinoma, sarcoma, leukemia, lymphoma and myeloma, and malignant tumors of the central nervous system.

At the present time, the leading therapies for cancer include surgery, radiation, and chemotherapy. Typically, surgery and radiation therapies are considered when cancer is locally confined. Existing chemotherapy treatments, while providing certain benefits, generally are disappointing in improving survival rates. Many of the cancer chemotherapy agents in clinical use are cytotoxins, which work by killing cells that exhibit rapid growth. Because these therapies kill cells that divide rapidly, normal cells that grow quickly, such as hair follicles, cells of the digestive tract, and bone marrow are also damaged or killed by cytotoxins, resulting in significant and often dangerous side effects to the patient, including hair loss, severe nausea, bone marrow depression, liver, heart and kidney damage, and immunosuppression.

For example, doxorubicin (hydroxydaunorubicin, also known as Adriamycin, “AMD” or “DOX”), which is an anthracycline antibiotic, is a commonly used chemotherapy drug in the treatment of a wide range of cancers, including hematological malignancies, many types of carcinoma, and soft tissue sarcomas. It is believed that, like other anthracyclines, doxorubicin works by intercalating DNA. Doxorubicin, however, is well documented to cause significant adverse effects, the most serious of which is life-threatening cardiotoxicity (heart damage), which is the dose-limiting toxicity of doxorubicin. Furthermore, bone marrow depression is dose-limiting for many cytotoxic drugs.

Thus, there is a continued unmet need for novel compounds, pharmaceutical compositions and methods of treatment that selectively kill cancer cells with minimal damage to normal cells. Such therapies would greatly increase not only the survival rates for cancer patients, but also significantly improve their quality of life during treatment.

Another major challenge to healthcare is inflammation, which plays a central role in many diseases and conditions such as rheumatoid arthritis, osteoarthritis, asthma, inflammatory bowel disease, rhinitis, conjunctivitis, dermatitis, cardiovascular diseases, atherosclerosis, Alzheimer's disease, as well as cancer. Inflammation is also a common cause of pain. Neutrophil granulocytes (or neutrophils) are the most abundant type of white blood cells in mammals and form an essential part of the essential immune system. Neutrophils are normally found in the blood stream and are one of the first-responders of inflammatory cells to migrate towards the site of inflammation.

Generally speaking, there are two classes of anti-inflammatory medicaments available today: steroids and NSAIDS. Steroid anti-inflammatory compounds, while potent and effective in the treatment of inflammatory diseases and conditions, cause numerous unfavorable side-effects, such as disturbance of carbohydrate metabolism, decreased calcium resorption, decreased excretion of endogenous corticosteroids and disturbance of physiological functions of the pituitary gland, adrenal cortex and thymus.

Thus, there is a continued unmet need for novel and effective anti-inflammatory compounds, pharmaceutical compositions and methods that are not accompanied by the many harmful side effects commonly associated with steroids and NSAIDS.

### DEFINITIONS

Definitions of specific functional groups and chemical terms are described in more detail below. General principles of organic chemistry, as well as specific functional moieties and reactivity, are described in “Organic Chemistry”, Thomas Sorrell, University Science Books, Sausalito: 2006.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are contemplated by the present invention. Those of ordinary skill in the art will readily appreciate that analogous ratios are contemplated for more complex isomer mixtures.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic methods well known in the art, and subsequent recovery of the pure enantiomers.

Given the benefit of this disclosure, one of ordinary skill in the art will appreciate that synthetic methods, as described herein, may utilize a variety of protecting groups. By the term “protecting group”, as used herein, it is meant that a particular functional moiety, e.g., O, S, or N, is temporarily blocked so that a reaction can be carried out selectively at another reactive site in a multifunctional compound. In preferred embodiments, a protecting group reacts selectively in good yield to give a protected substrate that is stable to the projected reactions; the protecting group should be selectively removable in good yield by preferably readily available, non-toxic reagents that do not attack the other functional groups; the protecting group forms an easily separable derivative (more preferably without the generation of new stereogenic centers); and the protecting group has a minimum of additional functionality to avoid further sites of reaction. Oxygen, sulfur, nitrogen, and carbon protecting groups may be utilized. Examples of a variety of protecting groups can be found in Protective Groups in Organic Synthesis, Third Ed. Greene, T.W. and Wuts, P.G., Eds., John Wiley & Sons, New York: 1999.

It will be appreciated that the compounds, as described herein, may be substituted with any number of substituents or functional moieties.

As used herein, the term “pharmaceutically acceptable salt” refers to either a pharmaceutical acceptable acid addition salt or a pharmaceutically acceptable base addition salt of a currently disclosed compound that may be administered without any resultant substantial undesirable biological effect(s) or any resultant deleterious interaction(s) with any other component of a pharmaceutical composition in which it may be contained.

As used herein, the term “pharmaceutically acceptable ester,” refers to esters that hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include formates, acetates, propionates, butyrates, acrylates and ethylsuccinates.

As used herein, (Cₓ-C_{y}) refers in general to groups that have from x to y (inclusive) carbon atoms. Therefore, for example, C₁-C₆ refers to groups that have 1, 2, 3, 4, 5, or 6 carbon atoms, which encompass C₁-C₂, C₁-C₃, C₁-C₄, C₁-C₅, C₂-C₃, C₂-C₄, C₂-C₅, C₂-C₆, and all like combinations. (C₁-C₂₀) and the likes similarly encompass the various combinations between 1 and 20 (inclusive) carbon atoms, such as (C₁-C₆), (C₁-C₁₂) and (C₃-C₁₂).

As used herein, the term “(Cₓ-C_{y})alkyl” refers to a saturated linear or branched free radical consisting essentially of x to y carbon atoms, wherein x is an integer from 1 to about 10 and y is an integer from about 2 to about 20. Exemplary (Cₓ-C_{y})alkyl groups include “(C₁-C₂₀)alkyl,” which refers to a saturated linear or branched free radical consisting essentially of 1 to 20 carbon atoms and a corresponding number of hydrogen atoms. Exemplary (C₁-C₂₀)alkyl groups include methyl, ethyl, n-propyl, isopropyl, n-butyl, isobutyl, dodecanyl, etc. Of course, other (C₁-C₂₀)alkyl groups will be readily apparent to those of skill in the art given the benefit of the present disclosure.

As used herein, the term, “(Cₓ-C_{y})alkoxy” refers to a straight or branched chain alkyl group consisting essentially of from x to y carbon atoms that is attached to the main structure via an oxygen atom, wherein x is an integer from 1 to about 10 and y is an integer from about 2 to about 20. For example, “(C₁-C₂₀)alkoxy” refers to a straight or branched chain alkyl group having 1-20 carbon atoms that is attached to the main structure via an oxygen atom, thus having the general formula alkyl-O—, such as, for example, methoxy, ethoxy, propoxy, isopropoxy, n-butoxy, sec-butoxy, tert-butoxy, pentoxy, 2-pentyl, isopentoxy, neopentoxy, hexoxy, 2-hexoxy, 3-hexoxy, and 3-methylpentoxy.

As used herein, the term “halogen” refers to fluorine (F), chlorine (Cl), bromine (Br), or iodine (I).

In general, the “effective amount” of an active agent refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the patient.

### SUMMARY OF THE INVENTION

The invention is based, in part, on the discovery of di-peptide conjugated antitumor agents, pharmaceutical compositions and methods for preparation and use thereof for treating various cancer and inflammation-related diseases and conditions. The invention addresses the shortcomings and inadequacies of the existing anti-tumor and anti-inflammatory drugs, particularly in that the anti-tumor agents of the invention selectively kill cancer cells with minimal damage to normal cells. Similarly, the anti-inflammatory agents of the invention provide effective treatment of various inflammatory diseases and conditions without the many deleterious side effects commonly associated with steroids and related treatments.

In one aspect, the invention generally relates to a compound of Formula I:

T-A₁-A₂-L-(X)ₙ (I)

or a pharmaceutically acceptable salt or ester thereof, wherein

T is a terminal group;

each of A₁ and A₂ is independently an amino-acid group;

L is a single bond,

wherein each R₁, R₂ and R₃ is independently a hydrogen, a C₁-C₆ alkyl group, a halogen, a C₁-C₆ alkoxy group; and

X is a group comprising an antitumor moiety, and n is 1 or 2,

with the proviso that when X is a doxorubicin group having the formula,

T is not a group selected from

In another aspect, the invention generally relates to a pharmaceutical composition comprising a compound of Formula I

T-A₁-A₂-L-(X)ₙ (I)

or a pharmaceutically acceptable salt or ester thereof, in an amount effective in the treatment or prevention of cancer, or a related disorder or condition thereof in a mammal, including a human, and a pharmaceutically acceptable carrier, wherein

T is a terminal group;

each of A₁ and A₂ is independently an amino-acid group;

L is a single bond,

wherein each R₁, R₂ and R₃ is independently a hydrogen, a C₁-C₆ alkyl group, a halogen, a C₁-C₆ alkoxy group; and

X is a group comprising an antitumor moiety, and n is 1 or 2.

In yet another aspect, the invention generally relates to a pharmaceutical composition comprising a compound of Formula I

T-A₁-A₂-L-(X)ₙ (I)

or a pharmaceutically acceptable salt or ester thereof, in an amount effective in the treatment or prevention of inflammation or a related disorder or condition thereof involving cathepsin B in a mammal, including a human, and a pharmaceutically acceptable carrier, wherein

T is a terminal group;

each of A₁ and A₂ is independently an amino-acid group;

L is a single bond,

wherein each R₁, R₂ and R₃ is independently a hydrogen, a C₁-C₆ alkyl group, a halogen, a C₁-C₆ alkoxy group; and

X is a group comprising an antitumor or anti-inflammatory moiety, and n is 1 or 2.

In yet another aspect, the invention generally relates to a method of treating or preventing cancer, or a related disorder or condition thereof in a mammal, including a human, comprising administering to a subject in need thereof a therapeutically effective amount of a pharmaceutical composition comprising a compound of Formula I

T-A₁-A₂-L-(X)ₙ (I)

or a pharmaceutically acceptable salt or ester thereof, effective in the treatment or prevention of cancer, or a related disorder or condition thereof in a mammal, including a human, and a pharmaceutically acceptable carrier, wherein

T is a terminal group;

each of A₁ and A₂ is independently an amino-acid group;

L is a single bond,

wherein each R₁, R₂ and R₃ is independently a hydrogen, a C₁-C₆ alkyl group, a halogen, a C₁-C₆ alkoxy group; and

X is a group comprising an antitumor moiety, and n is 1 or 2.

In yet another aspect, the invention generally relates to a method of treating or preventing inflammation, or a related disorder or condition thereof involving cathepsin B in a mammal, including a human, comprising administering to a subject in need thereof a therapeutically effective amount of a pharmaceutical composition comprising a compound of Formula I

T-A₁-A₂-L-(X)ₙ (I)

or a pharmaceutically acceptable salt or ester thereof, effective in the treatment or prevention of inflammation, or a related disorder or condition thereof in a mammal, including a human, and a pharmaceutically acceptable carrier, wherein

T is a terminal group;

each of A₁ and A₂ is independently an amino-acid group;

L is a single bond,

wherein each R₁, R₂ and R₃ is independently a hydrogen, a C₁-C₆ alkyl group, a halogen, a C₁-C₆ alkoxy group; and

X is a group comprising an antitumor or anti-inflammatory moiety, and n is 1 or 2.

In yet another aspect, the invention generally relates to a method of preventing, inhibiting or eliminating or cancer metastasis, in a mammal, including a human, comprising administering to a subject in need thereof a therapeutically effective amount of a pharmaceutical composition comprising a compound of Formula I

T-A₁-A₂-L-(X)ₙ (I)

or a pharmaceutically acceptable salt or ester thereof, effective in the inhibition or prevention of cancer in a mammal, including a human, and a pharmaceutically acceptable carrier, wherein

T is a terminal group;

each of A₁ and A₂ is independently an amino-acid group;

L is a single bond,

wherein each R₁, R₂ and R₃ is independently a hydrogen, a C₁-C₆ alkyl group, a halogen, a C₁-C₆ alkoxy group; and

X is a group comprising an antitumor moiety, and n is 1 or 2.

### BRIEF DESCRIPTION OF THE DRAWINGS

FIG. 1A shows an exemplary embodiment of the anti-tumor agents according to the invention, Ac-Phe-Lys-PABC-ADM (PADM), a conjugate of a dipeptide and ADM with the linkage, para-aminobenzyloxycarbonyl (PABC), a self-immolative spacer.

FIG. 1B shows exemplary in vitro data using SGC-7901 (gastric cancer) cells, demonstrating inhibition by PADM of cell growth in a dose-dependent fashion.

FIG. 2 shows effects of ADM and PADM on PC model.

FIG. 3 shows exemplary data on the impact of PADM and ADM on the general status of nude mice.

FIG. 4 shows exemplary data on the effect of PADM and ADM on liver and kidney functions.

FIG. 5 shows exemplary data on myocardium toxicity of PADM and ADM.

FIG. 6 shows exemplary data on Cat B expression in SGC-7901 tumor tissue.

FIG. 7 shows exemplary data in Table 1 on the effects of ADM and PADM on peripheral blood parameters.

### DETAILED DESCRIPTION OF THE INVENTION

The invention provides di-peptide conjugated antitumor agents, methods for their preparation, and pharmaceutical compositions and uses thereof, for treating various cancer and inflammation-related diseases and conditions.

The invention overcomes a number of deficiencies commonly seen in conventional anti-tumor and anti-inflammatory drugs and treatments. In particular, the invention offers novel and effective anti-tumor agents that selectively kill cancer cells with minimal or no damage to normal cells. Further, although most antitumor cytotoxic compounds are less effective against metastatic cells, the present invention has antimetastatic power greater than that of free drug because PDOX is unmasked by Cat B secreted by the cancer cells, and metastatic cells secrete more Cat B than the primary. Similarly, the anti-inflammatory compounds, pharmaceutical compositions and methods of the invention enable effective treatment of various inflammatory diseases and conditions without the many deleterious side effects commonly associated with steroids based treatments. Furthermore, the pharmaceutical agents methods of treatment disclosed herein are much more cost effective compared to the MAb-based immunoconjugate drugs.

The core approach of the invention in effectively treating cancer is to deliver a cytotoxic antitumor agent of the invention to the tumor cell but nowhere else by adding an inactivating chemical mask to the antitumor agent, which mask can be removed only by Cathepsin B (CB).

### Cancer and CB

The cathepsins are a family of cysteine proteases characterized by the presence of a cysteine residue in the catalytic site of the enzyme. They function in the normal physiological as well as pathological degradation of connective tissue. Cysteine proteases have been associated with a number of diseases and harmful conditions (e.g., arthritis, muscular dystrophy, inflammation, tumor invasion, glomerulonephritis, malaria, periodontal disease and atherosclerosis). Cathepsins play a major role in intracellular protein degradation, turnover and remodeling. Increased levels of CB, one of several known cathepsins, and redistribution of the enzyme are found in tumors, suggesting a role for cathepsin B in tumor invasion and metastasis. (Kos, et al. 1996 Oncology Reports 5: 1349-1361.) Aberrant CB activity is also connected to rheumatoid arthritis, osteoarthritis, atherosclerosis, pneumocystis carinii, acute pancreatitis, inflammatory airway disease and bone and joint disorders. Recent studies have also suggested that CB plays a pivotal role in Alzheimer's disease and other dementing conditions.

In the body, normally active CB occurs only within lysosomes and does not occur outside cells under normal conditions. Many types of cancer, however, secrete active CB on the outside of their cells. The secreted CB is not free to escape but rather bound to the plasma membrane. The amount of CB secreted increases with the degree of malignancy.

A small molecule conjugate of the invention, for example a di-peptide-doxorubicin conjugate, is stable in the body because there is normally no free CB to break it down and release free drug anywhere, including organs particularly attached by antitumor drugs: bone marrow, GI tract and heart. When the conjugate, during its random walk through the body, encounters the tumor, free CB on its perimeter removes the masking group and releases the free doxorubicin, which readily enters intact tumor cells and can kill or damage the tumor. The conjugate molecule remains intact anywhere else in the body due to the lack of CB to break down the masking group.

Previous studies have reported immunoconjugates that employed MAbs to conjugate with antitumor agent and guide the agent to the tumor. (See, e.g., U.S. Pat. No. 6,214,345 by Firestone, et al.) The distinction is the use of MAbs as a targeting guide. This distinction is important because such MAb-guided agents work in a fundamentally different way and rely exclusively on hitting cancer cells not because they display CB on the outside, but because they display a tumor antigen on the outside, to which the MAb bound. The agent is then internalized to lysosomes and cleaved there, and not on the cell surface, by CB.

It is well-known that activated neutrophils secrete CB, in addition to other destructive enzymes, at sites of inflammation such as atherosclerosis or rheumatoid arthritis, a painful and severely crippling disease. (Trabandt, et al. 1991 Arthritis and Rheumatism 34, 1444-1551; Lenarcic, et al. 1988 Biol. Chem. Hoppe-Seyler369 Suppl., 257-261; Codorean, et al. 1985 Morphologic and Embryologic 31, 269-274.)

In one aspect, the invention generally relates to a compound of Formula I:

T-A₁-A₂-L-(X)ₙ (I)

or a pharmaceutically acceptable salt or ester thereof, wherein

T is a terminal group;

each of A₁ and A₂ is independently an amino-acid group;

L is a single bond,

wherein each R₁, R₂ and R₃ is independently a hydrogen, a C₁-C₆ alkyl group, a halogen, a C₁-C₆ alkoxy group; and

X is a group comprising an antitumor moiety, and n is 1 or 2,

with the proviso that when X is a doxorubicin group having the formula,

T is not a group selected from

In certain preferred embodiments, A₂ is Lys and A₁ is Phe or Val with A₁ preferably being Phe.

In certain embodiments, X is

### and L is

And T, for example, may be selected from R—(C═O)— (wherein R is a C₁-C₆ alkyl), D-amino acid groups, trimethylated D-amino acid cations. For example, T may be D-Phe or trimethylated D-Phe.

In certain preferred embodiments, T is selected from t-butyloxycarbonyl (BOC), benzoyl, and phenylacetyl.

In certain preferred embodiments, T is selected a carbobenzoxy group having the formula:

wherein R₅ is one or more of a C₁-C₆ alkyl, a C₁-C₆ alkoxy group, halogen, —CN, methylsulfinyl, carbomethoxy, carboxy, dimethylamino, trimethlyammonio, and (m,p-CH₂OCH₂—).

Each R₁, R₂ and R₃ is independently a hydrogen, a C₁-C₆ alkyl group (e.g., methyl, ethyl, propyl, isopropyl, n-butyl, isobutyl), a halogen (e.g., F, Cl, Br, I), a C₁-C₆ alkoxy group (e.g., methoxy, ethyoxy, propoxy).

X may be any antitumor-active moiety, for example, those that are derived from an antitumor compound selected from Table 2. In certain embodiments, X is an antitumor-active moiety derived from an antitumor compound selected from anthracyclines, actinomycins, mitomycins, bleomycins, plicamycins.

TABLE 2
Antitumor Agents

| Alkylators | DNA intercalators | Nucleotide mimics |
|---|---|---|
| melphalan (L-PAM) | doxorubicin (adriamycin) | 5-FU (fluorouracil) |
| BCNU (Carmustine) | daunomycin | 6-mercaptopurine |
| CCNU (lomustine) | dapdox | 5-azacytidine |
| Me-CCNU | methoxymorpholino dox | 6-azauridine |
| Chlorambucil | 2-pyrrolamido dox | cytosine arabinoside |
| Mechlorethiminebusulfan | | 6-thioguanine |
| Cytoxan | | ara C |
| mitomycin C | | |
| neocarzinostatin | | |
| Antibiotics | Angiogenesis inhibitor | Folate mimics |
| streptozotocin | 17-amino-geldanamycin | methotrexate |
| dactinomycin | | |
| streptonigrin | | |
| Differentiation inducers | Fatty acids | |
| SAHA (suberoamido | bromopyruvic acid | |
| anilide hydroxamic | dichloroacetic acid | |
| acid) | 13-methyltetradecanoic | |
| Butyrate | acid | |
| phenylbutyrate | | |
| OSU-HDAC2 | | |
| Alkaloids | Mitosis interference | Others |
| actinomycin D | camptothecin | trimetrexate |
| vincristine | topotecan | carboplatin |
| vinblastine | CPT-11 | cis-platin |
| amsacrine | paclitaxel (taxol) | tetraplatin |
| gemcitabine | docetaxel | thio TEPA |
| bleomycin (tallysomycin) | BES (bisethylspermine) | etoposide (VP-16) |
| maytansine | difluoromethylornithine | teniposide (VM-26) |
| mithramycin | | colchicine |
| | | tamoxifen |
| | | hydroxyurea |
| | | sulforaphane |

In certain preferred embodiments, X is an antitumor-active moiety derived from doxorubicin, methotrexate (MTX), melphalan, mitomycin C, suberoylanilide hydroxamic acid (SAHA), fluorouracil (5-FU), camptothecin, paclitaxel, docetaxel, vincristine, bleomycin, tallysomycin and etoposide. Structures of exemplary antitumor agents are provided in Table 3.

```
TABLE 3
Structures of Select Antitumor Agents
  doxorubicin
  methotrexate (MTX)
  melphalan
  mitomycin C
  suberoylanilide hydroxamic acid (SAHA)
  fluorouracil (5-FU)
  camptothecin
  paclitaxel
  etoposide
  vincristine
  docetaxel
  tallysomycin
  Tallysomycin A
  Tallysomycin B
  bleomycin
```

In certain embodiments, n is 1 and L is

In certain preferred embodiments, n is 1 and L is

In certain embodiments, n is 1 and L is

In certain embodiments, n is 1 and L is a single bond.

In certain embodiments, n is 2 and L is

In another aspect, the invention generally relates to a pharmaceutical composition comprising a compound of Formula I

T-A₁-A₂-L-(X)ₙ (I)

or a pharmaceutically acceptable salt or ester thereof, in an amount effective in the treatment or prevention of cancer, or a related disorder or condition thereof in a mammal, including a human, and a pharmaceutically acceptable carrier, wherein

T is a terminal group;

each of A₁ and A₂ is independently an amino-acid group;

L is a single bond,

wherein each R₁, R₂ and R₃ is independently a hydrogen, a C₁-C₆ alkyl group, a halogen, a C₁-C₆ alkoxy group; and

X is a group comprising an antitumor moiety, and n is 1 or 2.

In certain preferred embodiments of the pharmaceutical composition, X is

### and L is

In certain preferred embodiments of the pharmaceutical composition, T is selected from t-butyloxycarbonyl (BOC), benzoyl, and phenylacetyl.

T can be a carbobenzoxy group having the formula:

wherein R₅ is one or more of a C₁-C₆ alkyl, a C₁-C₆ alkoxy group, halogen, —CN, methylsulfinyl, carbomethoxy, carboxy, dimethylamino, trimethlyammonio, and (m,p-CH₂OCH₂—).

The cancer to which the pharmaceutical composition of the invention is useful for treatment may be any cancer type, including bladder cancer, lung cancer, breast cancer, melanoma, colon and rectal cancer, non-Hodgkin lymphoma, endometrial cancer, ovarian cancer, gastric cancer, pancreatic cancer, kidney (renal cell) cancer, prostate cancer, leukemia, and thyroid cancer, for example.

In yet another aspect, the invention generally relates to a pharmaceutical composition comprising a compound of Formula I

T-A₁-A₂-L-(X)ₙ (I)

or a pharmaceutically acceptable salt or ester thereof, in an amount effective in the treatment or prevention of inflammation or a related disorder or condition thereof involving cathepsin B in a mammal, including a human, and a pharmaceutically acceptable carrier, wherein

T is a terminal group;

each of A₁ and A₂ is independently an amino-acid group;

L is a single bond,

wherein each R₁, R₂ and R₃ is independently a hydrogen, a C₁-C₆ alkyl group, a halogen, a C₁-C₆ alkoxy group; and

X is a group comprising an antitumor or anti-inflammatory moiety, and n is 1 or 2.

In certain embodiments of the pharmaceutical composition, the inflammation is selected from rheumatoid arthritis, osteoarthritis, atherosclerosis, inflammatory bowel disease, Crohn's disease, lupus erythematosis, type 1 diabetes, asthma, and myasthenia gravis. In certain preferred embodiments, the inflammation is selected from rheumatoid arthritis, osteoarthritis, and atherosclerosis.

In yet another aspect, the invention generally relates to a method of treating or preventing cancer, or a related disorder or condition thereof in a mammal, including a human, comprising administering to a subject in need thereof a therapeutically effective amount of a pharmaceutical composition comprising a compound of Formula I

T-A₁-A₂-L-(X)ₙ (I)

or a pharmaceutically acceptable salt or ester thereof, effective in the treatment or prevention of cancer, or a related disorder or condition thereof in a mammal, including a human, and a pharmaceutically acceptable carrier, wherein

T is a terminal group;

each of A₁ and A₂ is independently an amino-acid group;

L is a single bond,

wherein each R₁, R₂ and R₃ is independently a hydrogen, a C₁-C₆ alkyl group, a halogen, a C₁-C₆ alkoxy group; and

X is a group comprising an antitumor moiety, and n is 1 or 2.

In certain embodiments of the method, the cancer is selected from bladder cancer, lung cancer, breast cancer, melanoma, colon and rectal cancer, non-Hodgkin lymphoma, endometrial cancer, ovarian cancer, gastric cancer, pancreatic cancer, kidney (renal cell) cancer, prostate cancer, leukemia, and thyroid cancer. In certain preferred embodiments, the cancer is selected from breast cancer, lung cancer, colon cancer, ovarian cancer and gastric cancer. X may be an antitumor-active moiety derived from an antitumor compound selected from Table 2. In certain embodiments, X is an antitumor-active moiety derived from an antitumor compound selected Table 3.

In yet another aspect, the invention generally relates to a method of treating or preventing inflammation, or a related disorder or condition thereof involving cathepsin B in a mammal, including a human, comprising administering to a subject in need thereof a therapeutically effective amount of a pharmaceutical composition comprising a compound of Formula I

T-A₁-A₂-L-(X)ₙ (I)

or a pharmaceutically acceptable salt or ester thereof, effective in the treatment or prevention of inflammation, or a related disorder or condition thereof in a mammal, including a human, and a pharmaceutically acceptable carrier, wherein

T is a terminal group;

each of A₁ and A₂ is independently an amino-acid group;

L is a single bond,

wherein each R₁, R₂ and R₃ is independently a hydrogen, a C₁-C₆ alkyl group, a halogen, a C₁-C₆ alkoxy group; and

X is a group comprising an antitumor or anti-inflammatory moiety, and n is 1 or 2.

In certain embodiments of the method, the inflammation is selected from rheumatoid arthritis, osteoarthritis, atherosclerosis, inflammatory bowel disease, Crohn's disease, lupus erythematosis, type 1 diabetes, asthma, and myasthenia gravis. In certain preferred embodiments, the inflammation is selected from rheumatoid arthritis, osteoarthritis, and atherosclerosis.

In yet another aspect, the invention generally relates to a method of preventing, inhibiting or eliminating cancer metastasis, in a mammal, including a human, comprising administering to a subject in need thereof a therapeutically effective amount of a pharmaceutical composition comprising a compound of Formula I

T-A₁-A₂-L-(X)ₙ (I)

or a pharmaceutically acceptable salt or ester thereof, effective in the inhibition or prevention of cancer in a mammal, including a human, and a pharmaceutically acceptable carrier, wherein

T is a terminal group;

each of A₁ and A₂ is independently an amino-acid group;

L is a single bond,

wherein each R₁, R₂ and R₃ is independently a hydrogen, a C₁-C₆ alkyl group, a halogen, a C₁-C₆ alkoxy group; and

X is a group comprising an antitumor moiety, and n is 1 or 2.

In certain embodiments, the cancer is selected from bladder cancer, lung cancer, breast cancer, melanoma, colon and rectal cancer, non-Hodgkin lymphoma, endometrial cancer, ovarian cancer, gastric cancer, pancreatic cancer, kidney (renal cell) cancer, prostate cancer, leukemia, and thyroid cancer. In certain preferred embodiments, the cancer is selected from breast cancer, lung cancer, colon cancer, ovarian cancer and gastric cancer. X may be an antitumor-active moiety derived from an antitumor compound selected from Table 2. In certain embodiments, X is an antitumor-active moiety derived from an antitumor compound selected from Table 3.

In certain preferred embodiments, the pharmaceutical composition is administered to the subject for a period of time not less than 2 weeks immediately before a surgical operation to remove the primary cancer tissue. In certain preferred embodiments, the pharmaceutical composition is administered to the subject for a period of time not less than 2 weeks immediately after a surgical operation to remove the primary cancer tissue.

### Examples

### Example 1

### Ac-Phe-Lys-PABC-ADM for Treating Gastric Cancer

As one of the leading causes of cancer mortality in developing countries, gastric cancer trends to have lymphatic, haematogenous, or intra-abdominal metastasis due to its pathophysiological heterogeneity. Studies have shown that locoregional recurrence especially abdominal metastasis is the most common pattern of cancer recurrence, whether or not patients receive surgery alone or surgery combined with peri- or post-operative chemoradiotherapy. (Jemal, et al. 2011 CA Cancer J Clin. 61: 69-90; Macdonald, et al. 2001 N Engl J Med. 345: 725-730; Cunningham, et al. 2006 N Engl J Med. 355: 11-20; Sakuramoto, et al. 2007 N Engl J Med. 357: 1810-1820.) Gastric cancer can develop into intra-abdominal metastasis, mainly due to free cancer cells in peritoneal cavity. More than 30% advanced gastric cancer patients have developed peritoneal carcinomatosis when diagnosed, and 60% of all gastric cancer patients die of peritoneal carcinomatosis. (Yonemura, et al. 2009 J Surg Oncol. 100: 311-316; Yang, et al. 2011 Cytoreductive Surgery and Hyperthermic Intraperitoneal Chemotherapy Improves Survival of Patients with Peritoneal Carcinomatosis from Gastric Cancer: Final Results of a Phase III Randomized Clinical Trial. Ann Surg Oncol.)

During the development of peritoneal carcinomatosis, gastric cancer cells secret a host of enzymes to facilitate cancer cells seeding and colonization on the peritoneum. Cat B is one of the key enzymes in this critical process, over-expressed in gastric cancer as well as other cancers and actively involved in cancer invasion. (Dohchin, et al. 2000 Cancer 89: 482-487; Ebert, et al. 2005 Proteomics 5:1693-1704; Sitabkhan, et al. 2007 DNA Cell Biol. 26: 673-682; Eijan, et al. 2003 Cancer 98: 262-268; Czyzewska, et al. 2008 Folia Histochem Cytobiol. 46: 57-64; Sevenich, et al. 2010 Proc Natl Acad Sci USA 107: 2497-2502) On the other hand, Cat B is extremely low expressed in normal cells and inactive or lose activity as soon as it is dispersed in aqueous media away from cells. (Nouh, et al. 2011 J Trans' Med. 9:1; Atkinson, et al. 2008 Br J Pharmacol. 153: 1344-1352.)

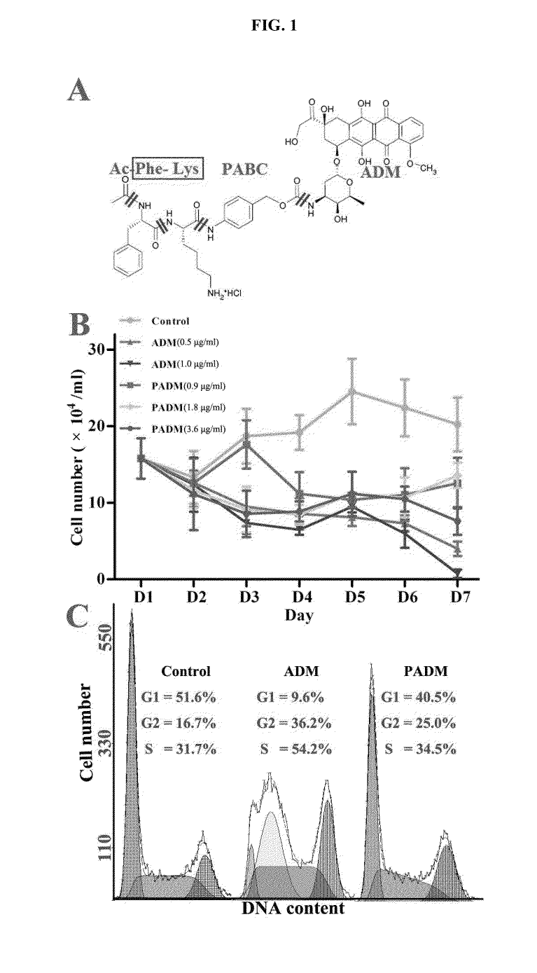

As shown in FIG. 1A, Ac-Phe-Lys-PABC-ADM (PADM) (hydrochloride) is a conjugate of a dipeptide and ADM with the linkage, para-aminobenzyloxycarbonyl (PABC), a self-immolative spacer. (Dubowchik, et al. 1998 Bioorg Med Chem Lett. 8: 3341-3346; Dubowchik, et al. 1998 Bioorg Med Chem Lett. 8: 3347-3352; 20. Dubowchik, et al. 2002 Bioconjug Chem. 13: 855-869.) The agent is inactive when there is little activated Cat B, such as normal tissues and peripheral blood, thus avoiding the side effects on normal tissue. During the intensive cancer invasion process, activated Cat B is over expressed in the exterior membrane of the invading cancer cells, which cleaves the Phe-Lys dipeptide at the Lys-PABC bond. Then, the exposed PABC spacer spontaneously solvolyzes and decarboxylates upon deacylation and free ADM molecules are released, resulting in direct killing of the invading cancer cells. The pure in vitro release study of PADM showed that the half-life of ADM release at 37° C. was 16 min in Cat B solution, but no changes were observed over 6-7 h in human plasma.

### Materials and Methods

Agents.

PADM was synthesized according to previously reported chemical process. (Dubowchik, et al. 1998 Bioorg Med Chem Lett. 8: 3341-3346; Dubowchik, et al. 1998 Bioorg Med Chem Lett. 8: 3347-3352; 20. Dubowchik, et al. 2002 Bioconjug Chem. 13: 855-869.) The molecular weight of PADM hydrochloride is 1045.50. In terms of equivalent mole content, 1.8 mg PADM hydrochloride is equivalent to 1 mg ADM hydrochloride (molecular weight 579.99). Other agents were obtained commercially, including Doxorubicin Hydrochloride for Injection (ADM) (Pharmacia, Milan, Italy) 10 mg/vial, RPMI-1640 medium (HyClone, NZ, USA) and Standard Newborn Bovine Serum (Zhengzhou Ben BioTech Co., Ltd., Zhengzhou, China) for cell culture, Propidine Iodide (PI) agents kit (Beckman coulter, CA, USA) for flow cytometric analysis, and rabbit anti-Cathepsin B polyclonal antibody (Lot No. 3190-100, BioVision, CA, USA) and peroxidase-conjugated Affinipure goat anti-rabbit IgG(H+L) (Lot No. 88813, Jackson ImmunoResearch, PA, USA) for immunohistochemical study.

Cell Lines.

Human gastric adenocarcinoma cell line SGC-7901 which had been widely used for gastric cancer research was maintained in Hubei Key Laboratory of Tumor Biological Behaviors. Cells were cultured in RPMI-1640 medium supplemented with 10% standard newborn bovine serum in the 5% CO2, saturated humidity, 37° C. incubator (Shel Lab, OR, USA). The culture medium was changed every 2 days.

Animals.

Male BALB/c nude mice, 5-6 weeks old were from Beijing HFK Bio-Technology Co. Ltd (animal quality certificate No. SCXK(Jing) 2009-0004) and maintained in an Animal Biosafety Level 3 Laboratory at the Animal Experimental Center of Wuhan University. After 3 days of adaptation, the animals were used for in vivo study, and the protocols were approved by the Animal Care Committee of Wuhan University.

### In Vitro Study

Cell Growth Assay.

SGC-7901 cells at exponential growth phase were planted in 24-well culture plates (Corning, N.Y., USA) at a density of 1×10⁵/well. After 48 h, cell numbers were determined by direct cell counting of 3 wells, which was designated as cell number of day 0 (D0). Then the cells were divided into 6 groups, treated with normal saline (100 μl), ADM (0.5 μg/ml), ADM (1.0 μg/ml), PADM (0.9 μg/ml), PADM (1.8 μg/ml), and PADM (3.6 μg/ml), respectively, 3 wells per group. Cells in each group were harvested and counted daily from D2 to D7. The cell growth curve was plotted.

Flow Cytometric Analysis.

To study the effect of PADM on cell cycle, flow cytometric analysis was performed. SGC-7901 cells were planted in 6-well culture plates (Corning, N.Y., USA), at a density of 2×10⁶/well. After 48 h, the cells were divided into 3 groups and treated for 24 h with normal saline (25 μl), ADM (0.25 μg/ml), and PADM (0.45 μg/ml). The cells in each group were harvested to make single-cell suspension, centrifuged at 4° C.×1000 rpm for 10 min, washed twice with phosphate buffered saline (pH=7.4), fixed and stained with PI kit. Cell cycle analysis was performed by flow cytometry with FC 500 (Beckman Coulter, Calif., USA).

### In Vivo Study

Pilot dosage study. Because this was the first time to conduct animal study, it was necessary to establish a workable dose range. Therefore, a pilot dosage study was first performed on 4 nude mice, which were divided into groups A (n=2) and B (n=2). The reported LD50 of ADM was 13.2 mg/kg when given intraperitoneally (i.p) and 12.0 mg/kg when given intravenously for mice. (He, et al. 2002 J Fourth Mil Med Univ. 23: 667-669; Kratz, et al. 2007 Hum Exp Toxicol. 26:19-35.) Therefore, this tentative study was performed based on these dosages. For group A, 24.0 mg/kg of PADM was used, which is equivalent to LD50 of ADM for i.p injection. The dosage of group B (36.0 mg/kg) was 1.5 times higher than that of group A. The general status of the mice was observed daily and body weight was recorded every 3 days. If the status of mice were stable in 7 days, additional administrations were given. After 4 consecutive administrations, the mice showed signs of toxicity. They were sacrificed and the heart, liver and kidney were obtained for histopathology study. The blood was used for biochemical studies, including cardiac, hepatic and renal functions.

GC Peritoneal Carcinomatosis Models and Therapeutic Study.

Based on the pilot dosage study, a total dosage of 57.6 mg/kg PADM was adopted for the full-scale animal study. SGC-7901 cells (5×10⁶/0.2 ml) were subcutaneously inoculated into the back of nude mice (n=4) as donor tumors, and cells (1×10⁷/0.4 ml) were i.p inoculated into the peritoneal cavity of nude mice (n=2). After 50 days, tumor nodules and ascites were obtained and tumor tissue homogenate was re-suspended in ascites at a density of 1×10⁷/ml (ascites were used as condition medium). These cell suspensions were injected i.p into 29 nude mice, 0.2 ml containing 5×10⁶ cells for each animal, on day 0 (D0). On D8, all of the mice were randomized into 3 groups: control group (n=9), ADM group (n=10) and PADM group (n=10). They were treated with normal saline (10 ml/kg), ADM (2.0 mg/kg) and PADM (7.2 mg/kg), respectively, on D8, D12, D16, D20, D24, D28, D32 and D36, respectively, by i.p injection. The total dosage of ADM was determined according to previous reports, and the dosage of PADM was determined according to the dosage of ADM and the results of pilot dosage study (7.2 mg/kg PADM was equivalent to 4 mg/kg ADM). (Schmid, et al. 2007 Bioconjug Chem. 18: 702-716.) The general status of mice was observed daily and body weight was recorded every 4 days. D40 was set as the end point of this study. On D16, D24 and D32, 80 μl of blood was obtained from tail veins, anticoagulated by EDTA, and analyzed by Sysmex KX-21 automated hematology analyzer (Sysmex, Kobe, Japan). On D40, all mice were sacrificed, blood was obtained, coagulated and centrifugated at 4° C.×3000 rpm for 10 min to separate serum for biochemical analysis, including ALT (alanine aminotransferase), AST (aspartate aminotransferase), BUN (blood urea nitrogen), Cr (creatinine), CK (creatine kinase), CK-MB (creatine kinase-MB), LDH (lactate dehydrogenase) by Aeroset Clinical Chemistry Analyzer (Abbott Laboratories, IL, USA).

### Histopathological Study

At autopsy, major organs including the heart, liver, kidneys, spleen and lungs were examined for any toxic changes. Any organs involved by the tumor and the tumor nodules were formalin-fixed, paraffin-embedded, and cut at 5-μm thickness, for histopathological study after hematoxylin and eosin (H&E) staining

### Immunohistochemical Study

In order to determine the Cat B level in this tumor model, immunohistochemical studies were performed on tumor tissue from control mice, following the detailed procedure developed in our group. (Peng, et al. 2011 Biomaterials 32:2907-2917.)

### Results

PADM is Shown Especially Potent on Metastatic Cells

TABLE 4
Percent inhibition of growth relative to controls

| | Dox | PADM | Ratio, PADM/Dox |
|---|---|---|---|
| Primary tumor | 42 | 44 | 1.05 |
| Metastases to: | | | |
| Mediastinal lymph nodes | 9.1 | 52 | 5.7 |
| Lung | 36 | 67 | 1.9 |
| Diaphragm | 19 | 44 | 2.3 |
| Mesenteric | 9.1 | 35 | 3.8 |
| Retroperitoneal | 65 | 79 | 1.2 |

PADM Showed Lower Inhibition on Cell Growth and Cell Cycle In Vitro.

As shown in FIG. 1B, both ADM and PADM could inhibit cell growth in a dose-dependent fashion, compared with control. At the end D7, the inhibition rates of ADM (0.5 μg/ml), ADM (1.0 μg/ml), PADM (0.9 μg/ml), PADM (1.8 μg/ml) and PADM (3.6 μg/ml) were 80.2%, 96.3%, 38.0%, 33.0%, and 62.3%, respectively. FIG. 1C showed ADM had significant effect on the cell cycle. In ADM treated cells, a prominent apoptosis peak was observed. In comparison, PADM treated cells did not show significant apoptosis, and the DNA content distribution was similar to that of control group.

PADM Had a Much Higher Maximum Tolerated Dose.

In pilot dosage study, the actual total dosage delivered was 96.0 mg/kg for group A, and 144.0 mg/kg for group B. There were no obvious changes in body weight among all the 4 animals for the first 3 injections. After the fourth administration (36 mg/kg for group A and 54 mg/kg for group B), however, persistent body weight decreases were observed. Based on these results, the ceiling dosage of below 96.0 mg/kg was set for formal in vivo test. No obvious damages were observed according to histopathological study of major organs.

PADM Retained an Even Better Antitumor Effect Compared to ADM.

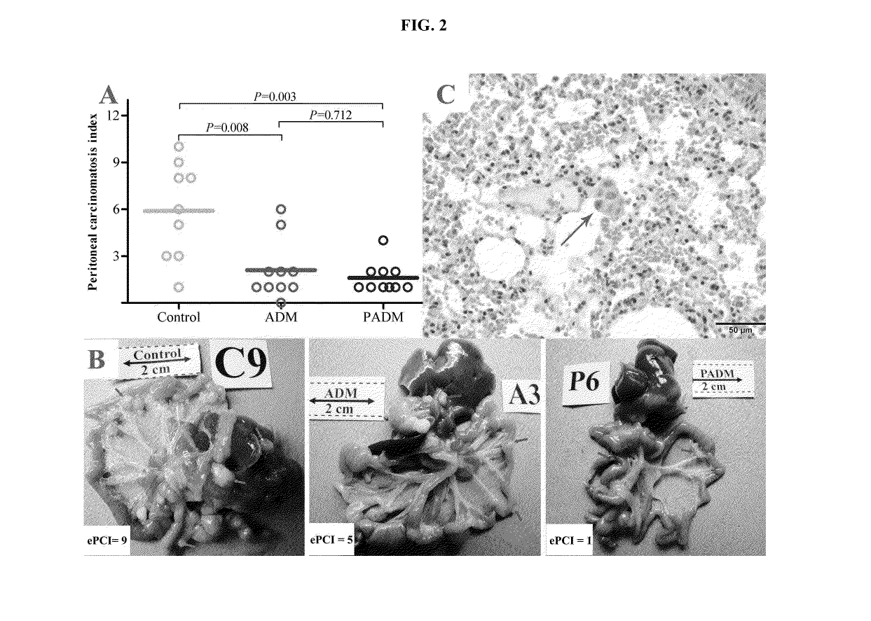

In the full-scale study, the actual total dosage of PADM (57.6 mg/kg) was 2 times of ADM (16 mg/kg) in terms of equal mole content. As shown in FIG. 2, Panel A shows the detailed ePCI score in each animal. Panel B shows representative pictures of peritoneal carcinomatosis in animals of control group (left, the 9th nude mouse), ADM group (middle, the 3rd nude mouse) and PADM group (right, the 6th nude mouse). Panel C shows lung metastasis in C9 nude mouse (HE stain, 200×, scale bar 50 μm).

Both PADM and ADM reduced the PC index, compared with control. The median (range) ePCI scores were 6 (1-10) for control group, 1.5 (0-6) for ADM group, and 1 (1-4) for PADM group, respectively (FIG. 2A, B). The difference in ePCI among the 3 groups was statistically significant (P=0.004), and ePCI of ADM and PADM groups were significantly lower than control group (P=0.008 and 0.003 respectively). There was no statistically significant difference in ePCI between ADM and PADM groups (P=0.712) (FIG. 2A). Lung metastasis was found in 1 nude mouse in the control groups (FIG. 2C), but there was no lung metastasis in any mouse of the PADM and ADM groups.

PADM Maintained Better General Status and Reduced General Toxicity Profiles.

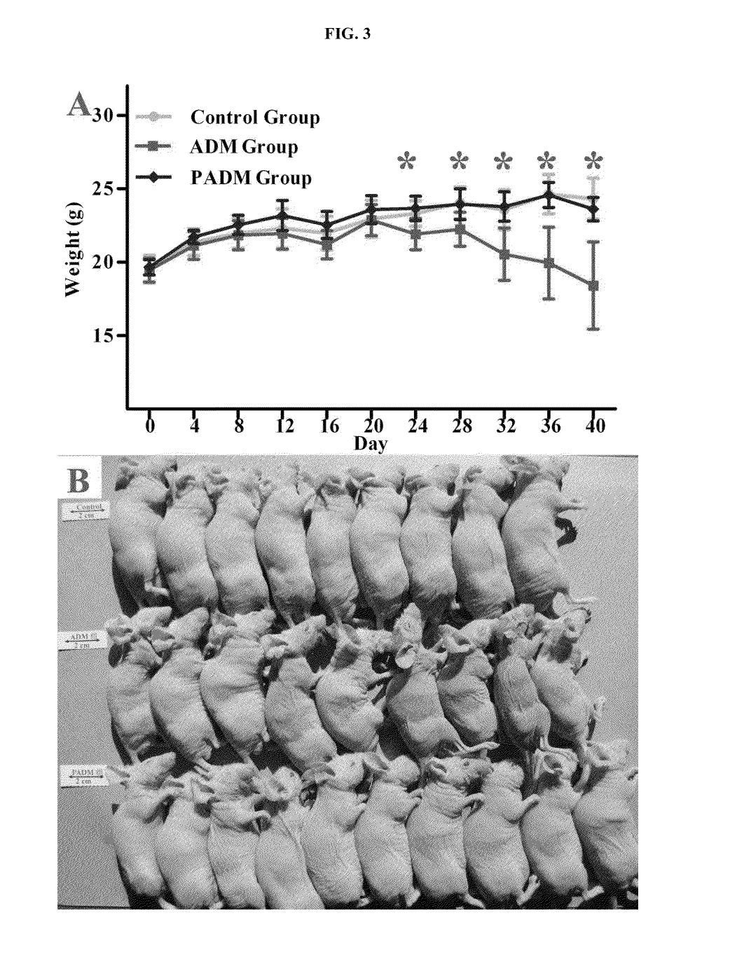

In terms of general status and body weight, PADM had significantly less negative impact (FIG. 3A, B). (A) Nude mice in PADM group had similar body weight to those of the control group throughout the whole study period. In comparison, nude mice in ADM group showed progressive decreases in body weight after 4 times of i.p ADM delivery. (B) Animal status at the study end point. Note 1 nude mouse in the ADM group died on D36 due to severe toxicity. * P<0.05.

The body weight of ADM group showed significant decrease on D24, and the drop became increasingly prominent thereafter. On the other hand, the body weight of PADM group was similar to that of control group throughout the whole study period.

The hematological effects of PADM and ADM were shown in Table 1. Of particular note was the consistent drop in red blood cells and lymphocytes across all 3 time points. ADM group had significantly lower RBC counts than control and the PADM groups. So was the lymphocytes count. On the other hand, there were no differences between PADM group and control group regarding these peripheral blood parameters.

PADM Reduced Toxicities on Liver, Kidney and Particularly the Heart.

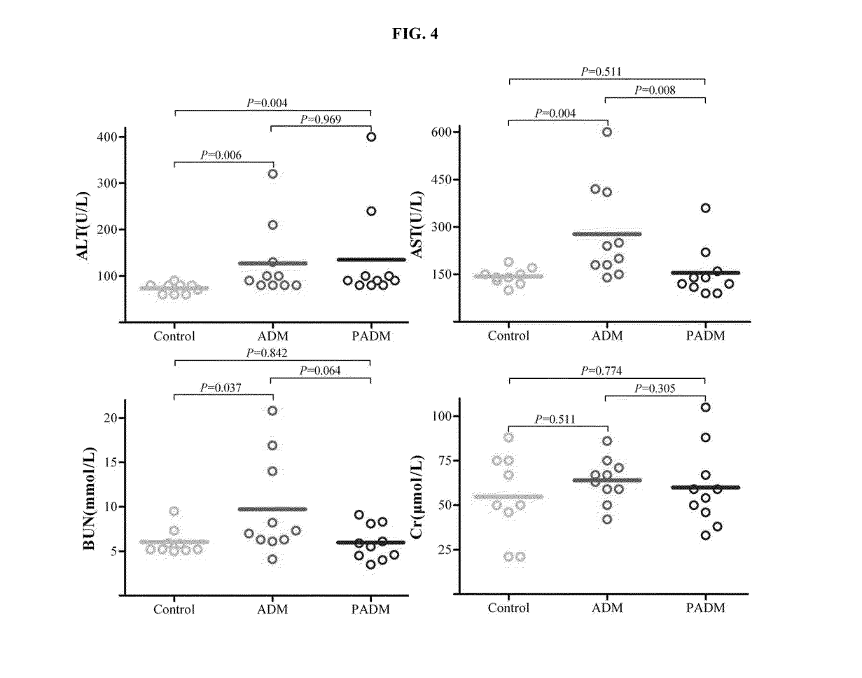

As shown in FIG. 4, both PADM and ADM had adverse effects on liver and kidney functions, but PADM was less toxic than ADM in terms of AST levels. For PADM, among the 4 major parameters studied, only ALT levels achieved statistical significance between the PADM and control groups. For ADM, however, 3 out of the 4 parameters achieved statistical significance between ADM and control groups. Histopathological study shown the same results that PADM did reduce the liver and renal toxicities.

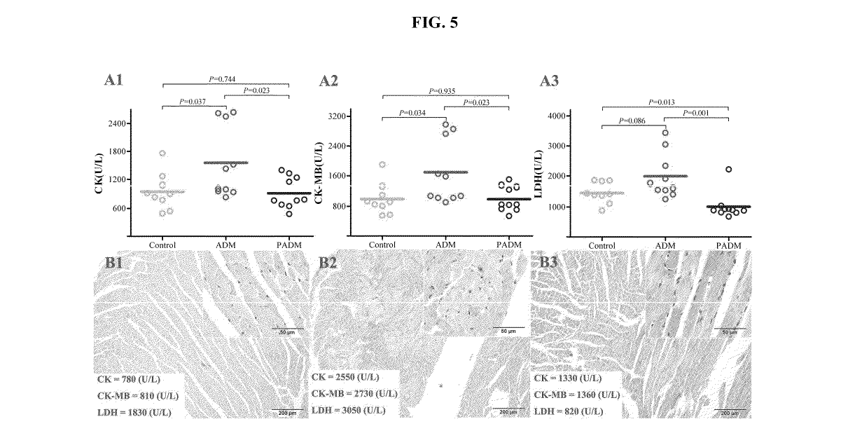

The potential cardiac toxicity was also studied in greater detail, as shown in FIG. 5. Three animals developed severe cardiac toxicities, as shown by significantly enzyme levels (A). Representative micrographs of myocardium in control (B1), ADM (B2) and PADM (B3) groups (B). Note the prominent degenerative changes of the myocardium in B2. (HE stain, 100×, Inserts are 400×). Histopathological studies found significant myocardium toxicities in 3, 7, 4 animals in control, ADM and PADM groups. The PADM group has significantly lower myocardium toxicities than the ADM group.

PADM Works Via the Cat B Pathway.

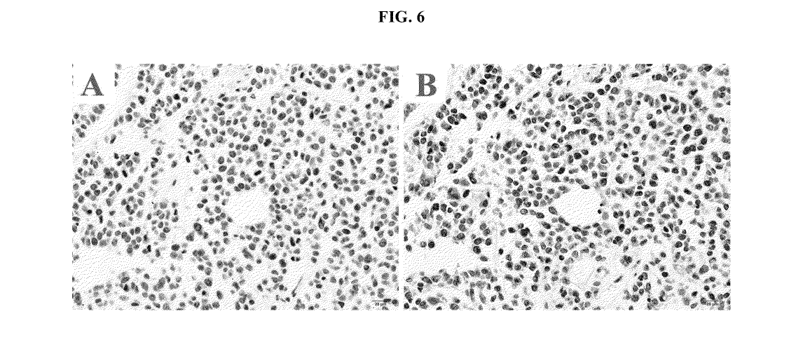

As shown in FIG. 6, prominent Cat B expression was observed in tumor cells, suggesting that PADM works via the Cat B pathway. AS shown, the enzymes were mainly expressed in the cytoplasm of tumor (A: negative control; B: ICH stain; 400×; Scale bar 20 μm). This is the first demonstration that PADM can kill tumor cells in vivo. PADM is indeed both stable and effective in vivo. Free ADM produced toxicity in the mice evidenced by weight loss beginning on D20, which was not seen in either the control or PADM groups up to D40. This shows that the amount of free ADM released from PADM by hydrolysis outside the tumor was small if any. However, though somatically stable, PADM released free ADM efficiently at the tumor, with antitumor power equivalent to that of free ADM.

All animals in the control group developed clinically significant peritoneal carcinomatosis, with tumor nodules seeding on the abdominal wall, diaphragm, liver capsule, small intestine surface and the mesenterium. Prominent angiogenesis was also observed in the abdominal tumor nodules. All these testify to the success of our model system. In order to more objectively evaluate efficacy, an ePCI system was established, similar to a clinical PCI system developed by Sugarbaker et al. and several rat ePCI systems. (Monneuse, et al. 2005 J Gastrointest Surg. 9: 769-774; Portilla, et al. 1999 World J Surg. 23: 23-29.) Compared with control, both PADM and ADM significantly reduced the ePCI. In terms of relative ePCI reduction, PADM and ADM reduced ePCI by 83.3% (5/6) and 75% (4.5/6) respectively. The results also suggest that both PADM and ADM had the same in vivo antitumor effect in this model system, where the ADM dosage of PADM was twice that of ADM.

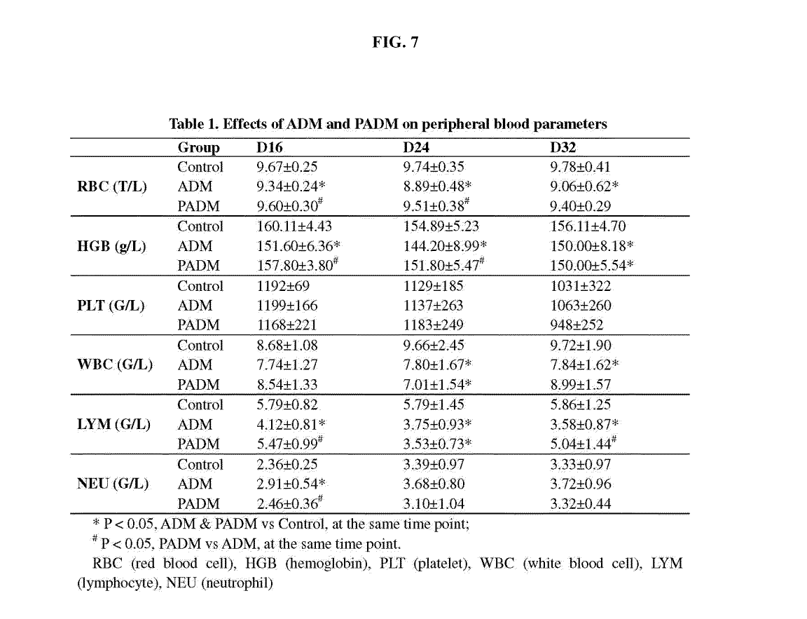

Also encouraging is the fact that the antitumor efficacy of PADM was accompanied by significantly improved general status, blood profiles and organ system tolerability. Animals in the PADM group maintained body weight throughout the study similar to those in the control group (23.61±0.80 g vs. 24.32±1.40 g), whereas there was significant body weight reduction in the ADM group beginning at the halfway point (18.40±2.97 g)(FIG. 3). This suggests that the overall side effects of PADM are much smaller than those of ADM, even though the dose of PADM was twice that of ADM. Besides the body weight benefit, the general status of the PADM group was also better. In terms of the routine peripheral blood test, both PADM and ADM had a negative effect on bone marrow function. However, PADM had a much smaller impact on red blood cells and lymphocytes than ADM (FIG. 7, Table 1). Thus the masking group apparently made PADM less toxic to the hemopoietic system. Biochemical studies also indicated that PADM had smaller toxicities to major organs such as the liver, the kidneys, and particularly the heart. In addition, histopathology showed prominent tissue structure and cell morphology changes, in agreement with these biochemical alterations. All these results indicate that the toxicity of PADM is much reduced by the Ac-Phe-Lys-PABC masking group.

To test whether PADM achieved its effects via the Cat B pathway, immunohistochemical studies were performed, which clearly showed that the SGC-7901 tumor indeed produced a large amount of Cat B. As was previously shown, 18 PADM was stable in human plasma, but rapidly cleaved in Cat B-rich medium. It can be inferred with reasonable confidence that in this animal model system, PADM inhibited PC development and progression through the Cat B mechanism.

Contents of the following paper is incorporated herein in its entirety for all purposes: “Cathepsin B cleavable novel prodrug Ac-Phe-Lys-PABC-ADM enhances efficacy at reduced toxicity in treating gastric cancer peritoneal carcinomatosis”. L-H Shiao, et al. 2012 Cancer 118[11], 2986.

In this specification and the appended claims, the singular forms “a,” “an,” and “the” include plural reference, unless the context clearly dictates otherwise.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

What is claimed is:

1. A compound of Formula I:
T-A₁-A₂-L-(X)ₙ (I)
or a pharmaceutically acceptable salt or ester thereof, wherein
T is a terminal group;
each of A₁ and A₂ is independently an amino-acid group;
L is a single bond,
wherein each R₁, R₂ and R₃ is independently a hydrogen, a C₁-C₆ alkyl group, a halogen, a C₁-C₆ alkoxy group; and
X is a group comprising an antitumor moiety, and n is 1 or 2,
with the proviso that when X is a doxorubicin group having the formula,
T is not a group selected from

2. The compound of claim 1, wherein A₂ is Lys and A₁ is Phe or Val.

3. (canceled)

4. The compound of claim 1, wherein X is
and L is

5. The compound of claim 4, wherein T is selected from t-butyloxycarbonyl (BOC), benzoyl, and phenylacetyl or a carbobenzoxy group having the formula:
wherein R₅ is one or more of a C₁-C₆ alkyl, a C₁-C₆ alkoxy group, halogen, —CN, methylsulfinyl, carbomethoxy, carboxy, dimethylamino, trimethlyammonio, and (m,p-CH₂OCH₂—).

6. (canceled)

7. The compound of claim 3, wherein X is an antitumor-active moiety derived from an antitumor compound selected from anthracyclines, actinomycins, mitomycins, bleomycins, plicamycins, or an antitumor compound selected from Table 2.

8. (canceled)

9. The compound of claim 1, wherein X is an antitumor-active moiety derived from doxorubicin, methotrexate (MTX), melphalan, mitomycin C, suberoylanilide hydroxamic acid (SAHA), fluorouracil (5-FU), camptothecin, paclitaxel, docetaxel, vincristine, bleomycin, tallysomycin and etoposide.

10. The compound of claim 1, wherein n is 1 and L is a single bond,

11. The compound of claim 1, wherein n is 2 and L is

12. (canceled)

13. A pharmaceutical composition comprising a compound of Formula I
T-A₁-A₂-L-(X)ₙ (I)
or a pharmaceutically acceptable salt or ester thereof, in an amount effective in the treatment or prevention of cancer, or a related disorder or condition thereof in a mammal, including a human, and a pharmaceutically acceptable carrier, wherein
T is a terminal group;
each of A₁ and A₂ is independently an amino-acid group;
L is a single bond,
wherein each R₁, R₂ and R₃ is independently a hydrogen, a C₁-C₆ alkyl group, a halogen, a C₁-C₆ alkoxy group; and
X is a group comprising an antitumor moiety, and n is 1 or 2.

14. The pharmaceutical composition of claim 13, wherein n is 1 and L is a single bond,

15. The pharmaceutical composition of claim 13, wherein n is 2 and L is

16. (canceled)

17. The pharmaceutical composition of claim 13, wherein A₂ is Lys and A₁ is Phe.

18. (canceled)

19. The pharmaceutical composition of claim 17, wherein X is
and L is

20. The pharmaceutical composition of claim 13, wherein T is selected from R—(C═O)— (wherein R is a C₁-C₆ alkyl), D-amino acid groups, trimethylated D-amino acid cations, t-butyloxycarbonyl (BOC), benzoyl, and phenylacetyl, and a carbobenzoxy group having the formula:
wherein R₅ is one or more of a C₁-C₆ alkyl, a C₁-C₆ alkoxy group, halogen, —CN, methylsulfinyl, carbomethoxy, carboxy, dimethylamino, trimethlyammonio, and (m,p-CH₂OCH₂—).

21. (canceled)

22. (canceled)

23. The pharmaceutical composition of claim 13, wherein the cancer is selected from bladder cancer, lung cancer, breast cancer, melanoma, colon and rectal cancer, non-Hodgkin lymphoma, endometrial cancer, ovarian cancer, gastric cancer, pancreatic cancer, kidney (renal cell) cancer, prostate cancer, leukemia, and thyroid cancer.

24. The pharmaceutical composition of claim 13, wherein X is an antitumor-active moiety derived from an antitumor compound selected from anthracyclines, actinomycins, mitomycins, bleomycins, plicamycins.

25. (canceled)

26. A pharmaceutical composition comprising a compound of Formula I
T-A₁-A₂-L-(X)ₙ (I)
or a pharmaceutically acceptable salt or ester thereof, in an amount effective in the treatment or prevention of inflammation or a related disorder or condition thereof involving cathepsin B in a mammal, including a human, and a pharmaceutically acceptable carrier, wherein
T is a terminal group;
each of A₁ and A₂ is independently an amino-acid group;
L is a single bond,
wherein each R₁, R₂ and R₃ is independently a hydrogen, a C₁-C₆ alkyl group, a halogen, a C₁-C₆ alkoxy group; and
X is a group comprising an antitumor or anti-inflammatory moiety, and n is 1 or 2.

27. The pharmaceutical composition of claim 26, wherein n is 1 and L is a single bond,

28. The pharmaceutical composition of claim 26, wherein n is 2 and L is

29. (canceled)

30. The pharmaceutical composition of claim 26, wherein A₂ is Lys and A₁ is Phe or Val.

31. (canceled)

32. The pharmaceutical composition of claim 26, wherein X is
and L is

33. The pharmaceutical composition of claim 26, wherein T is selected from R—(C═O)— (wherein R is a C₁-C₆ alkyl), D-amino acid groups, trimethylated D-amino acid cations, t-butyloxycarbonyl (BOC), benzoyl, and phenylacetyl and a carbobenzoxy group having the formula:
wherein R₅ is one or more of a C₁-C₆ alkyl, a C₁-C₆ alkoxy group, halogen, —CN, methylsulfinyl, carbomethoxy, carboxy, dimethylamino, trimethlyammonio, and (m,p-CH₂OCH₂—).

34. (canceled)

35. (canceled)

36. The pharmaceutical composition of claim 26, wherein the inflammation is selected from rheumatoid arthritis, osteoarthritis, atherosclerosis, inflammatory bowel disease, Crohn's disease, lupus erythematosis, type 1 diabetes, asthma, and myasthenia gravis.

37. A method of treating or preventing cancer, or a related disorder or condition thereof in a mammal, including a human, comprising administering to a subject in need thereof a therapeutically effective amount of a pharmaceutical composition comprising a compound of Formula I
T-A₁-A₂-L-(X)ₙ (I)
or a pharmaceutically acceptable salt or ester thereof, effective in the treatment or prevention of cancer, or a related disorder or condition thereof in a mammal, including a human, and a pharmaceutically acceptable carrier, wherein
T is a terminal group;
each of A₁ and A₂ is independently an amino-acid group;
L is a single bond,
wherein each R₁, R₂ and R₃ is independently a hydrogen, a C₁-C₆ alkyl group, a halogen, a C₁-C₆ alkoxy group; and
X is a group comprising an antitumor moiety, and n is 1 or 2.

38-46. (canceled)

47. The method of claim 37, wherein the cancer is selected from bladder cancer, lung cancer, breast cancer, melanoma, colon and rectal cancer, non-Hodgkin lymphoma, endometrial cancer, ovarian cancer, gastric cancer, pancreatic cancer, kidney (renal cell) cancer, prostate cancer, leukemia, and thyroid cancer.

48. (canceled)

49. (canceled)

50. A method of treating or preventing inflammation, or a related disorder or condition thereof involving cathepsin B in a mammal, including a human, comprising administering to a subject in need thereof a therapeutically effective amount of a pharmaceutical composition comprising a compound of Formula I
T-A₁-A₂-L-(X)ₙ (I)
or a pharmaceutically acceptable salt or ester thereof, effective in the treatment or prevention of inflammation, or a related disorder or condition thereof in a mammal, including a human, and a pharmaceutically acceptable carrier, wherein
T is a terminal group;
each of A₁ and A₂ is independently an amino-acid group;
L is a single bond,
wherein each R₁, R₂ and R₃ is independently a hydrogen, a C₁-C₆ alkyl group, a halogen, a C₁-C₆ alkoxy group; and
X is a group comprising an antitumor or anti-inflammatory moiety, and n is 1 or 2.

51-59. (canceled)

60. The method of claim 50, wherein the inflammation is selected from rheumatoid arthritis, osteoarthritis, atherosclerosis, inflammatory bowel disease, Crohn's disease, lupus erythematosis, type 1 diabetes, asthma, and myasthenia gravis.

61-76. (canceled)
